(19) United States
(12) Patent Application Publication
(10) Pub. No.: US 2026/0216538 A1
(43) Pub. Date: Jul. 30, 2026

(54) ULTRASOUND FAT REDUCTION EQUIPMENT AND METHOD OF USING THE SAME

(71) Applicant: LEADERMED GROUP US CORPORATION, Cranbury, NJ (US)

(72) Inventor: Qiao, Joanne JIANG CHEN, Pennington, NJ (US)

(73) Assignee: LEADERMED GROUP US CORPORATION, Cranbury, NJ (US)

(21) Appl. No.: 19/144,676

(22) PCT Filed: Dec. 6, 2023
(86) PCT No.: PCT/US2023/082620

§ 371 Date: Jun. 30, 2025

Related U.S. Application Data
(60) Provisional application No. 63/436,863, filed on Jan. 3, 2023.

(51) Int. Cl. A61N 7/00; G16H 40/63

(52) U.S. Cl. CPC A61N 7/00; G16H 40/63; A61N 2007/0008

(57) ABSTRACT

The disclosure includes an ultrasound fat reduction equipment and a method of using the same. The equipment comprises an input module, a storage module, a control and data processing module, and a machine learning module for using the data from the storage module to learn to generate a recommended best treatment procedure for a certain input data panel, and transferring to the control and data processing module to output the recommended best treatment procedure.

### BACKGROUND

In the field of aesthetic medicine, the most promising techniques for noninvasive body sculpting focus on ultrasound-induced fat reduction. These fat reduction ultrasound devices offer a practical way to reduce subcutaneous fat pads without significant adverse reactions.

Ultrasonic or ultrasound cavitation involves the use of ultrasound technology to break down fat cells located beneath the skin. This non-surgical approach is effective in reducing cellulite and localized fat deposits. During this procedure, ultrasonic vibrations are applied to exert energy on fat cells.

Various devices employ different methods. Some utilize high-frequency ultrasound waves to eliminate fat cells by generating heat or raising temperatures, while others use lower-frequency waves to mechanically disrupt the membranes of fat cells. Despite generally positive clinical outcomes, challenges such as non-responders, diminishing effects over time, inconsistent fat reduction results, and occasional side effects are common. Hence, there is a growing need for more effective non-invasive fat reduction equipment.

It's worth noting that individuals possess varying skin and fat structures and compositions, influenced by factors such as age, gender, and personal characteristics. As a result, there is a growing demand for personalized ultrasound treatments to cater to the unique needs and responses of each patient.

### SUMMARY OF THE INVENTION

The technical problem solved by this disclosure is providing an ultrasound fat reduction equipment to generate a recommended best treatment procedure for a certain input data panel for cavitating adipose tissue and for treating adipose in a region of interest.

In the first aspect, the ultrasound fat reduction equipment comprises:
an input module for inputting data;
a storage module for storing data from the input module;
a control and data processing module for controlling the generation of ultrasound which comprising turning on and turning off ultrasound, adjusting energy levels, and setting treatment durations, and processing data from the input module and the storage module; and
a machine learning module for using the data from the storage module to learn to generate a recommended best treatment procedure for a certain input data panel, and transferring to the control and data processing module to output the recommended best treatment procedure;
the input module, the storage module, and the machine learning module all communicate with the control and data processing module. In certain embodiments, the ultrasound fat reduction equipment further comprises one or more sensors, which is/are on the outer surface of the top of the ultrasound fat reduction equipment's handpiece; the sensor(s) is/are a pressure sensor, and/or a temperature sensor; the data using for the machine learning module includes individual characteristics data, treatment data, sensor data and the relationship of these 3 kinds of data.

In some embodiments, the machine learning module is configured to generate a recommended best treatment procedure through an AI model of optimized ultrasound cavitation treatment parameters.

In some embodiments, the ultrasound fat reduction equipment further comprises a movable transducer configured to generate ultrasound waves to cavitate fat cells and a first handpiece, and the movable transducer can move vertically inside the first handpiece, in order to change the distance between the movable transducer and the region of interest.

In some embodiments, the skin contacting end of the first handpiece's surface is textured with certain patterns, which are selected from closed design, open design and mixed design with both closed design and open design.

In some embodiments, the ultrasound fat reduction equipment further comprises a second handpiece, which can produce ultrasound waves using for ultrasound imaging.

In the second aspect, the disclosure also includes a method of using the ultrasound fat reduction equipment of the first aspect.

In some embodiments, the method of using the ultrasound fat reduction equipment comprises: inputting data which includes individual characteristics data; comparing the individual characteristics data with the data from a databank to generate a recommended best treatment procedure; wherein the databank is a part of the machine learning module and includes training set data and validation set data.

In some embodiments, the method of using the ultrasound fat reduction equipment comprises using coupling medium to fill the gap between a treatment head of the first handpiece and the region of interest; wherein said coupling medium is selected from composition A, and/or composition B, and/or composition C, and/or composition D; the composition A includes at least 1, 2, 3, 4, 5, 6, 7, 8, 9, 10, 11, 12, 13, 14 or all 15 of water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol, and methyl hydroxybenzoate; the composition B at least 1, 2, 3, 4, 5, 6, 7, 8, 9, or all 10 of includes water, propylene glycol, glycerin, carbomer, triethanolamine, p-hydroxyacetophenone, allantoin, dipotassium glycyrrhizinate, ribonucleic acid, and 1,2-pentanediol; the composition C includes at least 1, 2, 3, 4, 5, or all 6 of mineral oil, vitis vinifera seed oil, helianthus annus seed oil, citrus aurantium dulcis peel oil, tocopherol acetate, and hibiscus abelmoschus seed extract;
the composition D includes mRNA, and/or microRNA, and/or antisense RNA for collagen production.

During the ultrasound fat reduction treatment, the coupling medium gets absorbed into the skin and deliver many useful ingredients to tighten the skin. During treatment and the days after treatment, these ingredients will continue increase collagen production in skin cells at the treatment locations. In some embodiments, the ingredient includes mRNA, and/or microRNA, and/or antisense RNA for collagen production.

In the third aspect, the disclosure also includes a computer-readable medium, which comprises one or more processors for causing the processor to perform operations of the method of the second aspect.

In the forth aspect, the disclosure also includes an electronic device, which comprises: a processor, and a memory, the processor being connected to the memory;
the memory for storing a computer program of the processor;
wherein the processor is configured to implement the method described in the second aspect by executing the computer program.

According to the present disclosure, though the machine learning module the ultrasound fat reduction equipment can automatically personalize the treatment plan for each individual. With this feature the equipment can be used more widely and simply, which can even be used by some inexperienced operators but generate almost the same beneficial effect.

The feature of the movable transducer can provide several benefits:
1. Treatment Depth Control: The vertical movement of the transducer allows for control over the treatment depth. By adjusting the position of the transducer, the ultrasound fat reduction equipment can target different layers of fat or tissue, depending on the specific treatment objectives and the depth at which the cavitation effect is desired.
2. Customizable Treatment Areas: The ability to move the transducer up and down enables the treatment of different areas or contours of the body. It allows for flexibility in adapting to various body shapes and sizes, ensuring that the ultrasound energy is effectively delivered to the desired treatment areas.
3. Enhanced Treatment Precision: Precise vertical positioning of the transducer can improve treatment accuracy. It allows for better alignment with the targeted treatment area, minimizing the risk of unnecessary exposure to surrounding tissues and optimizing the effectiveness of the ultrasound waves.

The transducer, no matter is the movable transducer, the unmovable transducer, the first transducer or the second transducer, is a crucial component within the first handpiece that generates the ultrasound waves. The design may include a single transducer or an array of transducers depending on the specific machine. The shape and positioning of the transducer(s) within the first handpiece can affect the efficiency and targeting capabilities of the ultrasound energy.

The transducer converts electrical energy into mechanical vibrations that produce the ultrasound waves used for the cavitation process. As it consists of one or more piezoelectric elements. These elements, typically made of ceramic or crystal materials, possess the ability to deform when subjected to an electric field. This deformation generates the mechanical vibrations necessary for ultrasound wave generation.

### BRIEF DESCRIPTION OF THE DRAWINGS

FIG. 1 shows the surface of the skin contacting end of the first handpiece with a mixed design (DESIGN 1), consisting of ovals and arcs, when viewed from the top.

FIG. 2 shows the surface of the skin contacting end of the first handpiece with a mixed design (DESIGN 2), consisting of ring and arcs, when viewed from the top.

FIG. 3 shows the surface of the skin contacting end of the first handpiece with a rings design (DESIGN 3) when viewed from the top.

FIG. 4 shows the surface of the skin contacting end of the first handpiece with an ovals design (DESIGN 4) when viewed from the top.

FIG. 5 shows the surface of the skin contacting end of the first handpiece with a wavy design (DESIGN 5) when viewed from the top.

FIG. 6 shows the surface of the skin contacting end of the first handpiece with a parallel design (DESIGN 6), consisting of some straight parallel lines when viewed from the top.

FIG. 7 shows the surface of the skin contacting end of the first handpiece with a parallel design (DESIGN 7), consisting of some arcs parallel lines and a straight line in the middle, when viewed from the top.

FIG. 8 shows the surface of the skin contacting end of the first handpiece with a radiant design (DESIGN 8), consisting of some straight lines, when viewed from the top.

FIG. 9 shows the surface of the skin contacting end of the first handpiece with a radiant design (DESIGN 9), consisting of some wavy lines, when viewed from the top.

FIG. 10 shows the surface of the skin contacting end of the first handpiece with an open design (DESIGN 10), consisting of some rings with openings, when viewed from the top.

FIG. 11 shows the surface of the skin contacting end of the first handpiece with an open design (DESIGN 11), consisting of some ovals with openings, when viewed from the top.

FIG. 12 shows the surface of the skin contacting end of the first handpiece with a raised rings design (DESIGN 3) when viewed from the side.

FIG. 13 shows the surface of the skin contacting end of the first handpiece with a raised mixed design (DESIGN 1), consisting of ovals and arcs, when viewed from the side.

FIG. 14 shows the surface of the skin contacting end of the first handpiece with a raised radiant design (DESIGN 8), consisting of some straight lines, when viewed from the side.

FIG. 15 shows the surface of the skin contacting end of the first handpiece with a concave rings design (DESIGN 3) when viewed from the side.

FIG. 16 shows the surface of the skin contacting end of the first handpiece with a concave mixed design (DESIGN 1), consisting of ovals and arcs, when viewed from the side.

FIG. 17 shows the surface of the skin contacting end of the first handpiece with a concave radiant design (DESIGN 8), consisting of ovals and arcs, when viewed from the side.

FIG. 18 shows the surface of the skin contacting end of the first handpiece with a raised wavy design (DESIGN 5), consisting of some straight lines, when viewed from the side.

FIG. 19 shows the surface of the skin contacting end of the first handpiece with a concave wavy design (DESIGN 5), consisting of some straight lines, when viewed from the side.

FIG. 20 shows schematic diagram of the communication of modules of the ultrasound fat reduction equipment in some embodiments.

FIG. 21 shows schematic diagram of the handpiece comprising a movable transducer in some embodiments.

FIG. 22 shows schematic diagram of the first handpiece comprising a first transducer and a second transducer in some embodiments.

FIG. 23 shows schematic diagram of the first handpiece comprising sensors in some embodiments with sensors flushing with the outer surface of the treatment head of the first handpiece.

FIG. 24 shows schematic diagram of the first handpiece comprising sensors in some embodiments with membrane structure of sensors.

### DETAILED DESCRIPTION OF THE INVENTION

The technical solution of the present invention will be further described below with reference to the accompanying drawings and specific embodiments.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

The disclosure includes an ultrasound fat reduction equipment using non-focused ultrasound low frequency waves and method of using the same.

In the first aspect, the ultrasound fat reduction equipment comprises:
an input module for inputting data;
a storage module for storing data from the input module;
a control and data processing module for controlling the generation of ultrasound which comprising turning on and turning off ultrasound, adjusting energy levels, and setting treatment durations, and processing data from the input module and the storage module; and
a machine learning module for analyzing the data from the storage module to generate a model so it can recommend best treatment procedure for a certain input data panel, and transferring to the control and data processing module to output the recommended best treatment procedure;
the input module, the storage module, and the machine learning module all communicate with the control and data processing module.

In some embodiments, the ultrasound fat reduction equipment further comprises a display module for displaying, and the display module communicates with the control and data processing module.

In some embodiments, the ultrasound fat reduction equipment further comprises one or more sensors, which is/are on the outer surface of the top of the ultrasound fat reduction equipment's handpiece; the sensor(s) is/are a pressure sensor, and/or a temperature sensor; the data using for the machine learning module includes individual characteristics data, treatment data, sensor data and the relationship of these 3 kinds of data.

In some embodiments, the individual characteristics data includes at least 1, 2, 3, 4, 5, 6, 7, 8, 9, 10, or all 11 of age, gender, weight, BMI waist measurement, fat content in %, fat layer thickness, density of the fat in the treatment area, degree of skin tightness or looseness, and ultrasound images; the treatment data includes at least 1, 2, 3, or all 4 of energy levels, treatment durations, ultrasound frequencies and treatment outcome data; the sensor data includes data from the pressure sensor, and/or data from the temperature sensor.

In some embodiments, the control and data processing modules are also used to change the distance between the movable transducer and the region of interest on commands from the input module or the control and data processing module itself.

In some embodiments, the machine learning module is configured to generate a recommended best treatment procedure through an AI model of optimized ultrasound cavitation treatment parameters. That is there is an artificial intelligence (AI) model of optimized ultrasound cavitation treatment parameters in the machine learning module. The modeling process of the AI model of optimized ultrasound cavitation treatment parameters is as follows:
1. Data Collection: Gather patients' personal characteristic data (the individual characteristics data) from multiple sources, including but not limited to age, gender, weight, body mass index (BMI), waist measurement, fat content in %, fat layer thickness, density of the fat in the treatment area, degree of skin tightness or looseness, ultrasound images, skin type, target area, medical history, medication, sensitivity/pain tolerance, and lifestyle factors. Additionally, collect a database containing ultrasound cavitation treatment parameters (energy levels, treatment durations, ultrasound frequencies, and mode of ultrasound, such as continuous or pulsed waves), corresponding treatment outcome data, and sensor data (data from a pressure sensor, and data from a temperature sensor).
2. Data Preprocessing: Preprocess and clean the collected data, including data format conversion, outlier detection, and handling missing values. Ensure the accuracy and completeness of the data.
3. Feature Engineering: Extract meaningful features from patients' personal characteristic data and perform appropriate feature transformation and scaling. This may involve techniques such as feature selection, principal component analysis (PCA), or other methods.
4. Data Set Partitioning: Divide the dataset into training, validation, and testing sets. The training set is used for constructing the AI model, the validation set is used for parameter tuning and model selection, and the testing set is used to evaluate the model's performance.
5. AI Model Construction: Select suitable AI algorithms, such as machine learning algorithms or deep learning neural networks and train the model using the training set. Depending on the dataset and objectives, supervised learning, unsupervised learning, or reinforcement learning approaches can be employed.
6. Parameter Optimization: Use the AI model and training dataset for parameter optimization to find the optimal ultrasound cavitation treatment parameters. This can be achieved through optimization algorithms such as gradient descent, genetic algorithms, or Bayesian optimization.
7. Model Evaluation: Evaluate the model's performance on the validation set using different parameter combinations. This involves comparing the model's predictions with the actual outcomes in the validation set and calculating various performance metrics such as accuracy, recall, and F1 score.
8. Model Adjustment and Selection: Adjust and optimize the model based on the results from the validation set to find the best model parameters and ultrasound cavitation treatment parameter combinations.
9. Parameter Recommendation: Utilize the trained AI model to provide personalized recommendations for ultrasound cavitation treatment parameters for new patients, aiming to achieve optimal treatment outcomes.
10. Model Deployment: Deploy the trained and validated AI model into real clinical practice and continuously optimize it using real-time patient data for validation and improvement.

There are various AI modeling methods that can be utilized in the context of ultrasound cavitation treatment parameter optimization. Some of the typical AI modeling methods include:
1. Supervised Learning: This approach involves training a model using labeled data, where the input data is paired with corresponding target labels. The model learns the mapping between the input features (patients' personal characteristics) and the desired output (optimized treatment parameters). Common supervised learning algorithms include linear regression, logistic regression, support vector machines (SVM), and random forests.
2. Unsupervised Learning: Unsupervised learning aims to discover patterns or structures in the data without explicit target labels. It can be useful for exploring relationships within the dataset and identifying clusters or subgroups of patients with similar characteristics. Clustering algorithms like k-means clustering and hierarchical clustering, as well as dimensionality reduction techniques like principal component analysis (PCA) and t-SNE, fall under unsupervised learning methods.
3. Deep Learning: Deep learning is a subset of machine learning that involves training artificial neural networks with multiple layers. Deep learning models, such as convolutional neural networks (CNN) and recurrent neural networks (RNN), have shown remarkable success in various domains, including image recognition, natural language processing, and medical data analysis. These models can be leveraged to extract intricate patterns and relationships from complex ultrasound cavitation treatment datasets.
4. Reinforcement Learning: Reinforcement learning focuses on training an agent to make sequential decisions in an environment to maximize a reward signal. While less commonly used in ultrasound cavitation parameter optimization, reinforcement learning could potentially be employed to optimize treatment parameters over time based on feedback and outcomes. Algorithms like Q-learning and policy gradients are commonly used in reinforcement learning.

5. Causal Inference: Causal inference methods aim to understand the causal relationships between variables and identify the causal effects of certain interventions or treatments. These methods can be valuable in determining the causal impact of specific treatment parameters on patient outcomes. Techniques like propensity score matching, instrumental variables, and causal graphical models are commonly employed in causal inference.

Causal inference is a branch of statistics and data analysis that aims to understand and estimate the causal relationships between variables. It seeks to answer questions about cause-and-effect relationships rather than just correlations or associations between variables. In the context of ultrasound cavitation treatment parameter optimization, causal inference can help determine the causal impact of specific treatment parameters on patient outcomes.

Causal inference methods often involve inferring causality from observational data, as conducting randomized controlled trials (RCTs) may not always be feasible or ethical in certain scenarios. These methods rely on assumptions and statistical techniques to estimate causal effects based on observational data.

There are several commonly used approaches in causal inference.
1. Propensity Score Matching: Propensity score matching is a technique used to balance the distribution of confounding variables between treatment groups. It involves estimating the propensity score, which is the probability of receiving a particular treatment given the observed covariates. By matching or weighting individuals based on their propensity scores, researchers can estimate the causal effect of the treatment parameter of interest while minimizing the influence of confounding variables.
2. Instrumental Variables: Instrumental variables (IV) are used to estimate causal effects in the presence of unobserved confounders. An instrumental variable is a variable that is associated with the treatment parameter but has no direct effect on the outcome, except through its impact on the treatment. By using instrumental variables, researchers can identify the causal effect of the treatment parameter of interest by exploiting the variation explained by the instrumental variable.
3. Causal Graphical Models: Causal graphical models, such as Bayesian networks or directed acyclic graphs (DAGs), provide a graphical representation of causal relationships between variables. These models help visualize and understand the causal structure of a system, allowing researchers to identify the direct and indirect effects of treatment parameters on patient outcomes. Causal graphical models can be used to guide the estimation of causal effects and assess the sensitivity of the results to potential confounding factors.

Causal inference methods aim to provide more rigorous and reliable insights into the causal relationships between ultrasound cavitation treatment parameters and patient outcomes. However, it's important to note that causal inference heavily relies on the assumptions made during the analysis, and care must be taken to ensure these assumptions are valid and appropriate for the specific context and data available.

Machine learning using causal inference can be a powerful approach for optimizing ultrasound cavitation treatment parameters based on patients' personal characteristics and outcome data while considering causal relationships. Here shows how machine learning using causal inference can be applied:
1. Define the Causal Model: Develop a causal model that represents the relationships between patients' personal characteristics, ultrasound cavitation treatment parameters, and outcome variables. This model should include causal assumptions and variables of interest. Techniques like causal graphical models (e.g., Bayesian networks) can help visualize and formalize these relationships.
2. Data Collection: Gather a dataset that includes patients' personal characteristics, ultrasound cavitation treatment parameters, and corresponding outcome data. Ensure the data captures the relevant variables and reflects a diverse population to obtain reliable causal estimates.
3. Causal Effect Estimation: Apply causal inference methods to estimate the causal effects of ultrasound cavitation treatment parameters on patient outcomes. Techniques such as propensity score matching, instrumental variables, or structural equation modeling can be employed depending on the nature of the data and research question.
4. Model Training: Utilize machine learning algorithms within the causal inference framework to train a model that predicts optimal ultrasound cavitation treatment parameters based on patients' personal characteristics while considering the estimated causal effects. This may involve combining supervised learning techniques with causal inference methods.
5. Validation and Evaluation: Assess the performance and robustness of the trained model using appropriate evaluation metrics. Validate the model using cross-validation techniques or hold-out validation on new patient data to ensure its generalizability.
6. Treatment Parameter Optimization: Utilize the trained model to recommend optimized ultrasound cavitation treatment parameters for new patients based on their personal characteristics. The model should account for the estimated causal effects, allowing for informed decisions regarding parameter adjustments to achieve desired outcomes.
7. Monitoring and Continuous Improvement: Continuously monitor the performance and effectiveness of the model in real-world settings. Incorporate new data and update the model as necessary to improve its accuracy and keep up with evolving treatment practices.

Machine learning using causal inference allows for a deeper understanding of the causal relationships between patients' characteristics, ultrasound cavitation treatment parameters, and outcomes. By incorporating causal inference methods into the machine learning pipeline, the model can provide optimized treatment parameter recommendations while considering the underlying causal mechanisms.

There are several other machine learning methods that can be used for optimizing ultrasound cavitation treatment parameters based on patients' personal characteristics and outcome data. Some commonly employed techniques include:
1. Regression Analysis: Traditional regression analysis methods, such as linear regression, logistic regression, or decision trees, can be used to model the relationship between patients' characteristics and treatment outcomes. These models can help identify important predictors and their impact on the desired treatment parameters.
2. Gradient Boosting: Gradient boosting algorithms, such as XGBoost or LightGBM, are powerful machine learning methods that sequentially build an ensemble of weak prediction models. They optimize a loss function by adding new models that correct the errors made by previous models. Gradient boosting can capture complex interactions and handle large datasets effectively.
3. Gaussian processes (GPs): GPs are probabilistic models that can capture complex relationships and uncertainties in data. GPs can be used to model the relationship between patients' characteristics and treatment outcomes and provide probabilistic predictions. They are particularly useful when dealing with small datasets or when uncertainty estimation is critical.

The treatment parameters can be considered as input data in machine learning models for ultrasound cavitation treatment parameter optimization. These treatment parameters represent the settings or configurations of the ultrasound reduction equipment during the treatment process. They can include variables such as:
1. Frequency: The frequency of the ultrasound waves used in the cavitation treatment. It can be specified in kilohertz (kHz).
2. Intensity: The intensity or power level of the ultrasound waves applied during the treatment. It can be measured in units such as watts per square centimeter (W/cm²).
3. Duration: The duration or length of time the ultrasound cavitation treatment is applied to the target area. It can be measured in seconds or minutes.
4. Mode: The specific mode or pattern of ultrasound application, such as continuous or pulsed waves.
5. Energy Delivery: The method or technique used to deliver the ultrasound energy to the target area, which can vary depending on the specific ultrasound cavitation device.
6. Transducer positions: ?

These treatment parameters can be used as input features in machine learning models alongside patients' personal characteristics and outcome data. The model learns the relationships between these parameters, patients' characteristics, and the corresponding treatment outcomes to optimize the ultrasound cavitation treatment for individual patients. By analyzing the patterns and correlations in the data, the model can recommend optimal treatment parameter settings that are likely to achieve the desired outcomes based on patients' specific characteristics.

In some embodiments, the ultrasound fat reduction equipment for cavitating adipose tissue and for treating adipose in a region of interest, comprises a movable transducer configured to generate ultrasound waves to break fat cells and a first handpiece; the movable transducer can move up and down inside the first handpiece, in order to change the distance between the movable transducer and the region of interest.

In some embodiments, the ultrasound fat reduction equipment further comprises one or more rods inside of the first handpiece; the movable transducer is configured to slide along the rod(s) by a mechanical switch pushing control on the surface of the first handpiece or by electronical control to change the distance between the movable transducer and the region of interest.

In some embodiments, the distance between the movable transducer and the region of interest is arranged from 0.5-5.0 cm.

In some embodiments, the movable transducer comprises:
a first transducer configured to generate ultrasound waves with lower frequency, in the range of 25-50 kHz, preferably is 25-45 kHz, 30-45 kHz, 30-40 kHz, more preferably is 35 kHz or 40 kHz; and/or
a second transducer configured to generate ultrasound waves with higher frequency, in the range of 50-90 kHz, preferably is 55-90 kHz, 55-70 kHz, more preferably is 60 KHz.

In some embodiments, the number of the first transducer is one, and the number of the second transducers is more than one, preferably is 2-10 or 2-6 or 2-3, and the second transducers are configured to surround the first transducer.

In some embodiments, the ultrasound fat reduction equipment further comprises an unmovable transducer configured to generate ultrasound waves with lower frequency, in the range of 25-50 kHz, preferably is 25-45 kHz, 30-50 kHz, 30-45 kHz, or 30-40 kHz, more preferably is 35 kHz or 40 kHz; the movable transducer is configured to generate ultrasound waves with higher frequency, in the range of 50-90 kHz, preferably is 55-90 kHz, 55-85 kHz, or 55-70 kHz, more preferably is 60 kHz.

In some embodiments, the ultrasound fat reduction equipment further comprises an unmovable transducer configured to generate ultrasound waves with higher frequency, in the range of 50-90 kHz, preferably is 55-85 kHz, 55-70 kHz, more preferably is 60 kHz; the movable transducer is configured to generate ultrasound waves with lower frequency, in the range of 25-45 kHz, preferably is 30-45 kHz, 30-40 kHz, more preferably is 35 or 40 kHz.

In some embodiments, the ultrasound fat reduction equipment further comprises one or more pressure sensors, which is/are on the outer surface of the top of the first handpiece.

In some embodiments, the pressure sensor(s) is/are wired sensor(s) or wireless sensor(s).

In some embodiments, the ultrasound fat reduction equipment further comprises one or more temperature sensors, which is/are on the outer surface of the top of the first handpiece.

In some embodiments, the temperature sensor(s) is/are wired sensor(s) or wireless sensor(s).

In some embodiments, the ultrasound fat reduction equipment further comprises a cooling system, an acoustic focusing mechanism, and a housing. The cooling system includes heat sinks and/or cooling fans to dissipate excess heat generated by a transducer (it can be the movable transducer, the unmovable transducer, the first transducer and/or the second transducer). The heat sinks and the cooling fans are inside of the first handpiece.

The acoustic focusing mechanism helps concentrate the ultrasound waves to a specific treatment area, improving the precision and effectiveness of the cavitation process. The acoustic focusing mechanism is also inside of the first handpiece and is between the transducer and the treatment head of the first handpiece.

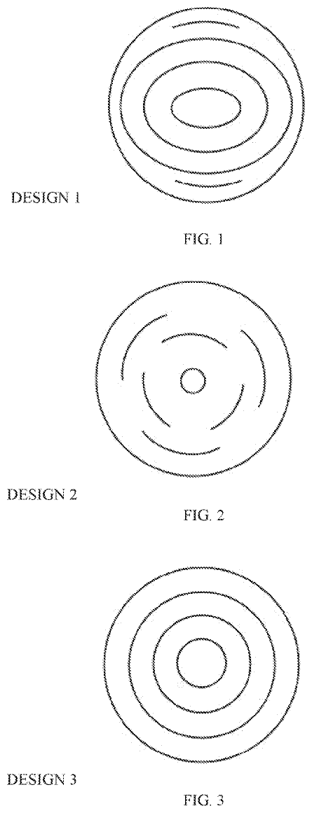

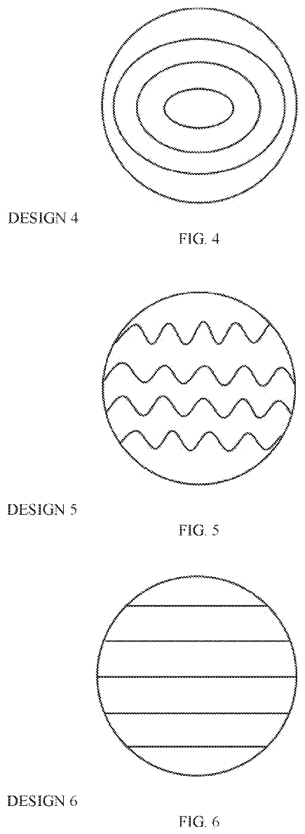

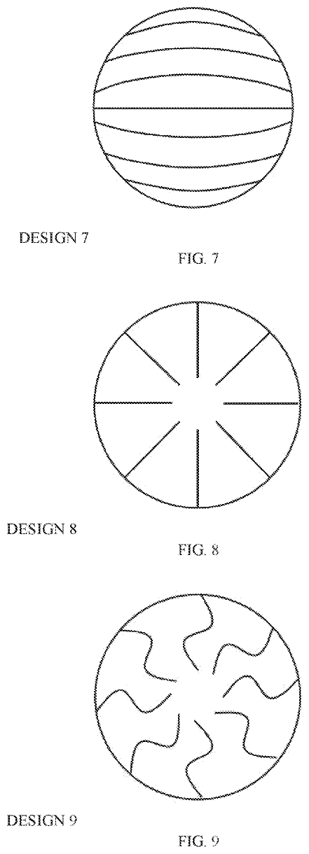

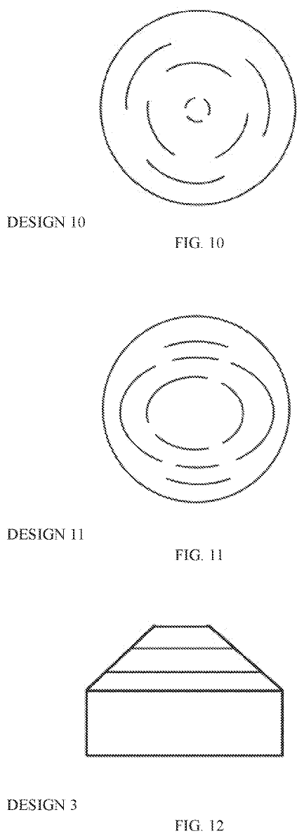

In some embodiments, the surface of the skin contacting end of the first handpiece has textured with certain patterns so that it will potentially have enhanced energy distribution, reduced hotspots, improved contact, aesthetic appeal, ease of handling, and reduced acoustic reflection. The certain patterns are selected from closed design, open design and mixed design (e.g. FIGS. 1 and 2) with both closed design and open design. The closed design includes one or more of ring design (e.g. FIG. 3) and oval design (e.g. FIG. 4). The open design includes one or more of wavy design (e.g. FIG. 5), parallel design (e.g. FIGS. 6 and 7), radiant design (e.g. FIGS. 8 and 9), ring with openings design (e.g. FIG. 10), and oval with openings design (e.g. FIG. 11).

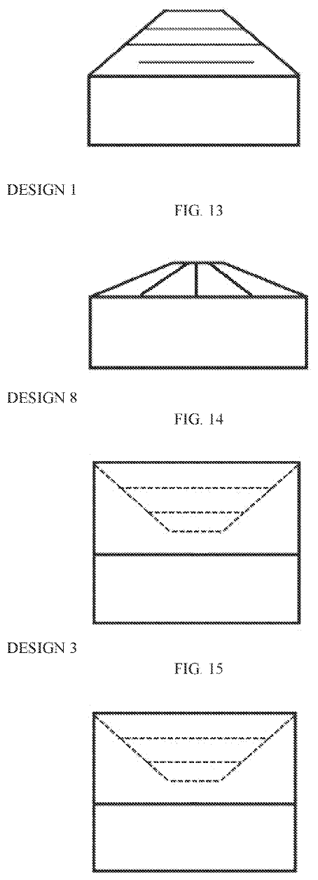

In some embodiments, the surface of the skin contacting end is raised, preferably the center of the surface is raised, more preferably the center of the surface is gradually raised (e.g. FIG. 12 to 14).

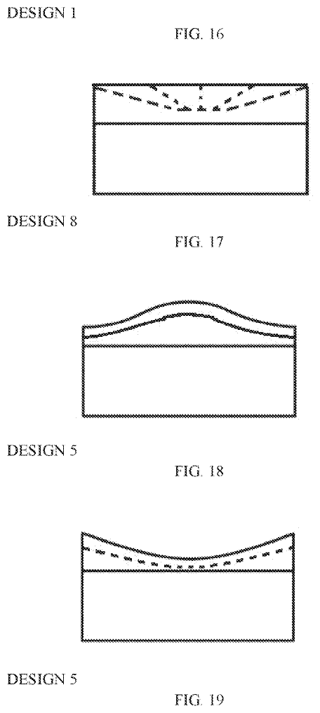

In some embodiments, the surface of the skin contacting end is concave, preferably with a concavity in the center of the surface, more preferably with a gradual concavity in the center of the surface (e.g. FIG. 15 to 17).

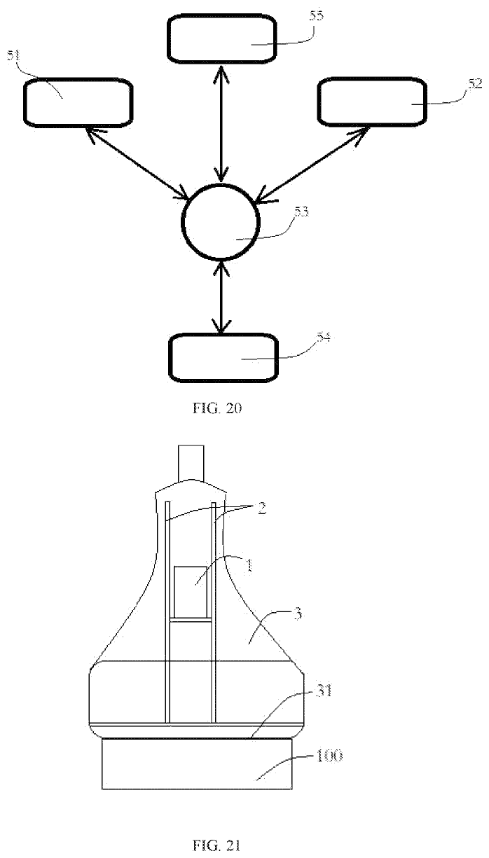

In some embodiments, the surface of the skin contacting end is height variant, such as the center wavy line of the wavy design is raised higher than the side wavy lines of it (e.g. FIG. 18), or the center wavy line of the wavy design is concave deeper than the side wavy lines of it (e.g. FIG. 19).

FIG. 1 to 11 show the top view of DESIGNS 1 to 11, respectively. These DESIGNS are raised or concave when viewed from the side. For example, FIG. 12 to 14 and FIG. 18 show that DESIGN 3, DESIGN 1, DESIGN 8, and DESIGN 5 are raised, respectively. FIG. 15 to 17 and FIG. 19 show that DESIGN 3. DESIGN 1, DESIGN 8, and DESIGN 5 are concave, respectively.

In some embodiments, the surface of the skin contacting end is height variant, such as the center parallel line of the parallel design is raised higher than the side parallel lines of it, or the center parallel line of the parallel design is raised lower than the side parallel lines of it.

In some embodiments, the ultrasound fat reduction equipment further comprises an ear protection element, such as earplugs, earmuffs, noise-canceling earbuds, noise-canceling headphones, and custom-made ear protection.

In some embodiments, the noise-canceling earbuds and/or the noise-canceling headphones are selected from Bose QuietComfort Earbuds, Sony WF-1000XM4, Apple AirPods Pro and Jabra Elite 85t.

In some embodiments, the ultrasound fat reduction equipment further comprises a second handpiece, which can produce ultrasound waves using for ultrasound imaging. The ultrasound fat reduction equipment further comprises an ultrasound cavitation module and an ultrasound imaging module. The first handpiece is belonged to the ultrasound cavitation module, and the second handpiece is belonged to the ultrasound imaging module.

The ultrasound cavitation module involves the application of low-frequency ultrasound waves to the targeted area of the body. These ultrasound waves create microbubbles in the fat tissue, causing them to expand and contract rapidly. The continuous expansion and contraction of these bubbles generate pressure changes within the fat cells, leading to their breakdown. The broken-down fat cells release their contents, which are then metabolized and eliminated by the body's natural processes.

The ultrasound imaging module is a component that uses ultrasound waves to create images of structures within the body. It works by emitting high-frequency sound waves that bounce off internal structures and return as echoes. These echoes are then used to create real-time images of organs, tissues, and other structures. The ultrasound imaging module has an ultrasound imaging transducer inside of the second handpiece. The ultrasound imaging transducer is capable of emitting and receiving ultrasound waves for real-time imaging.

The ultrasound fat reduction equipment further comprises a control interface and an imaging display. The control interface would allow the operator to control both the cavitation and imaging functions, and to select which function to use and adjust the corresponding parameters. The corresponding parameters could include settings for adjusting energy levels, treatment duration, and imaging parameters.

The imaging display is a screen to display the real-time ultrasound images captured by the ultrasound imaging module. This display would aid the operator in visualizing the treatment area during the procedure, such as fat deposits, muscles, and other tissues. With the ultrasound imaging module's guidance, the operator can identify the specific fat deposits to be targeted for the cavitation procedure. The operator can adjust the position and orientation of the treatment device based on the imaging feedback to ensure precise targeting. With the ultrasound imaging module, the operator can monitor the effects of the cavitation in real-time, ensuring that the fat cells are being effectively broken down while minimizing impact on surrounding tissues. The operator can use post-treatment imaging to assess the immediate effects of the procedure and ensure uniform treatment coverage.

Workflow: The operator's workflow with a dual-function machine would involve the following steps:

### 1. Imaging Phase

The operator selects the imaging function on the control interface.

The imaging handpiece (the second handpiece) is used to scan the treatment area, capturing real-time ultrasound images.

### 2. Targeting and Planning

Based on the imaging feedback, the operator identifies the areas for fat reduction and plans the treatment approach.

### 3. Cavitation Phase

The operator switches to the cavitation function on the control interface.

The cavitation handpiece (the first handpiece) is used to apply the low-frequency ultrasound waves to the targeted fat deposits.

### 4. Real-Time Monitoring

Throughout the cavitation procedure, the operator can switch between two modules and periodically.

The operator can monitor the treatment progress and effects on fat cells in real-time.

### 5. Evaluation and Documentation

After completing the cavitation procedure, the operator can use the imaging handpiece to assess the immediate effects and ensure uniform treatment coverage.

The captured images can also be used for documentation and patient records.

Skin Type: The patient's skin type classification (e.g., Fitzpatrick scale), which can affect how the ultrasound energy is absorbed and transmitted through the skin.

Target Area: The specific area of the body being targeted for the ultrasound cavitation treatment, as different body parts may have varying tissue properties and treatment requirements.

Medical History: Relevant medical history of the patient, including any pre-existing conditions, allergies, or previous treatments that may impact the suitability or effectiveness of the ultrasound cavitation treatment.

Medications: Information about any medications or supplements the patient is currently taking, as certain medications can interact with the treatment or affect the healing process.

Sensitivity/Pain Tolerance: The patient's sensitivity or pain tolerance level, which can influence the intensity or duration of the treatment.

Lifestyle Factors: Factors such as physical activity level, smoking status, and dietary habits that may affect treatment outcomes and the overall success of the ultrasound cavitation treatment.

The data (pressure data) from a pressure sensor can affect the transmission of ultrasound waves and their interaction with the target tissue. By monitoring and incorporating the pressure as an input feature, the machine learning module can account for its impact on treatment outcomes. This information can help optimize the treatment parameters to ensure an appropriate and consistent level of pressure for effective and safe results.

Monitoring the temperature during the treatment by the temperature sensor can provide valuable insights into the thermal effects of ultrasound cavitation. It can help assess whether the temperature remains within a safe and effective range to avoid potential tissue damage. By including temperature measurements as input data, the machine learning module can learn the relationship between temperature changes and treatment outcomes. This knowledge can guide the optimization of treatment parameters to achieve the desired therapeutic effects while maintaining a suitable temperature range, and also can guide the control and data processing module to control the cooling system to lower the temperature.

In the second aspect, the disclosure also includes a method of using the ultrasound fat reduction equipment of the first aspect.

In some embodiments, the method of using the ultrasound fat reduction equipment comprises: inputting data which includes individual characteristics data; comparing the individual characteristics data with the data from a databank to generate a recommended best treatment procedure; wherein the databank is a part of the machine learning module and includes training set data and validation set data.

In some embodiments, the method of using the ultrasound fat reduction equipment comprises using coupling medium to fill the gap between a treatment head of the first handpiece and the region of interest; wherein said coupling medium is selected from composition A, and/or composition B, and/or composition C, and/or composition D; the composition A includes at least 1, 2, 3, 4, 5, 6, 7, 8, 9, 10, 11, 12, 13, 14 or all 15 of water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol, and methyl hydroxybenzoate; the composition B at least 1, 2, 3, 4, 5, 6, 7, 8, 9, or all 10 of includes water, propylene glycol, glycerin, carbomer, triethanolamine, p-hydroxyacetophenone, allantoin, dipotassium glycyrrhizinate, ribonucleic acid, and 1,2-pentanediol; the composition C includes at least 1, 2, 3, 4, 5, or all 6 of mineral oil, vitis vinifera seed oil, helianthus annus seed oil, citrus aurantium dulcis peel oil, tocopherol acetate, and hibiscus abelmoschus seed extract; the composition D includes mRNA, and/or microRNA, and/or antisense RNA for collagen production.

During the ultrasound fat reduction treatment, the coupling medium gets absorbed into the skin and deliver many useful ingredients to tighten the skin. During treatment and the days after treatment, these ingredients will continue increase collagen production in skin cells at the treatment locations. In some embodiments, the ingredient includes mRNA, and/or microRNA, and/or antisense RNA for collagen production.

In some embodiments, the composition A further includes at least 1, 2, 3, 4, 5, or all 6 of xanthan gum, dipotassium glycyrrhizinate, essence, ethylhexylglycerol, EDTA disodium, and glucose.

In some embodiments, the composition B further includes at least 1, 2, 3, 4, 5, 6, 7, 8, 9, 10, 11, 12, 13, 14, 15, 16, 17, 18, or all 19 of hydroxyethylcellulose, octyl hydroxamic acid, sodium hyaluronate, punica granatum peel extract, nonylphenol polyether-15, butanediol, essence, nonylphenol polyether-10, camellia sinensis extract, glycerol octanoate, ellagic acid, galla rhois extract, 1,2-hexanediol, glutathione, polysorbate-60, disodium hydrogen phosphate, acetylhexapeptide-8, and sodium dihydrogen phosphate.

In the third aspect, the disclosure also includes a computer-readable medium, which comprises one or more processors for causing the processor to perform operations of the method of the second aspect.

In the forth aspect, the disclosure also includes an electronic device, which comprises: a processor, and a memory, the processor being connected to the memory:
the memory for storing a computer program of the processor;
wherein the processor is configured to implement the method described in the second aspect by executing the computer program.

### First Embodiment

In this embodiment, the ultrasound fat reduction equipment for cavitating adipose tissue and for treating adipose in a region of interest comprises a movable transducer, two rods, some sensors and a first handpiece.

The ultrasound fat reduction equipment further comprises:
an input module 51 for inputting data;
a storage module 52 for storing data from the input module 51;
a control and data processing module 53 for controlling the generation of ultrasound which comprising turning on and turning off ultrasound, adjusting energy levels, and setting treatment durations, and processing data from the input module 51 and the storage module 52;
a display module 54 for displaying; and
a machine learning module 55 for using the data from the storage module 52 to learn to generate a recommended best treatment procedure for a certain input data panel, and transferring to the control and data processing module 53 to output the recommended best treatment procedure;
the input module 51, the storage module 52, the display module 54, and the machine learning module 55 all communicate with the control and data processing module 53 (FIG. 20).

The data using for the machine learning module includes individual characteristics data, treatment data, sensor data and the relationship of the 3 kinds of data; the individual characteristics data includes age, gender, weight, BMI, waist measurement, fat content in %, fat layer thickness, density of the fat in the treatment area, degree of skin tightness or looseness; the treatment data includes energy levels, treatment durations, ultrasound frequencies and treatment outcome data; the sensor data includes data from a pressure sensor, and data from a temperature sensor.

The movable transducer is configured to slide along the rods by electronical control to change the distance between the movable transducer and the region of interest. The electronical control represents that the change of the distance between the movable transducer and the region of interest is controlled by the control and data processing module 53. As a result that the control and data processing module 53 is further for controlling to change the distance between the movable transducer and the region of interest by order from the input module or the control and data processing module itself.

The movable transducer comprises a first transducer and a second transducer The first transducer configured to generate ultrasound wave with lower frequency, in the range of 25-45 kHz. The second transducer configured to generate ultrasound wave with higher frequency, in the range of 55-85 kHz. The number of the first transducer is one, and the number of the second transducers is 6. The second transducers are in a circle, and the first transducer is in the center of the circle.

The sensors are wired pressure sensors (they can be wired temperature sensors, or can be wired pressure sensors and wired temperature sensors, in some embodiments). The detection interface of the wired pressure sensors are flush with the outer surface of the treatment head of the first handpiece. The wire of the wired pressure sensors gets through the tail of the first handpiece to connect to the control and data processing module of the ultrasound fat reduction equipment.

### Second Embodiment

In this embodiment (FIG. 21), the ultrasound fat reduction equipment for cavitating adipose tissue and for treating adipose in a region of interest 100 comprises a movable transducer 1, two rods 2, and a first handpiece 3. The movable transducer 1 is configured to slide along the rods 2 by a mechanical switch pushing control on the surface of the first handpiece to change the distance between the movable transducer 1 and the region of interest 100. The movable transducer 1 and the two rods 2 are inside the first handpiece 3. The two rods 2 are vertical to the top of the first handpiece. The top of the first handpiece also known as the treatment head 31 of the first handpiece.

### Third Embodiment

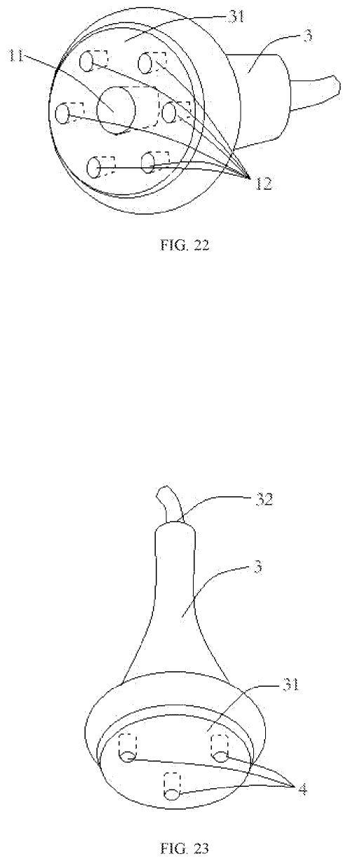

In this embodiment (FIG. 22), the ultrasound fat reduction equipment for cavitating adipose tissue and for treating adipose in a region of interest comprises a movable transducer, two rods, and a first handpiece 3. The movable transducer is configured to slide along the rods by electronical control to change the distance between the movable transducer and the region of interest.

The movable transducer comprises a first transducer 11 and a second transducer 12. The first transducer 11 configured to generate ultrasound wave with lower frequency, in the range of 25-45 kHz, preferably is 30-40 kHz, more preferably is 35 kHz. The second transducer 12 configured to generate ultrasound wave with higher frequency, in the range of 55-85 kHz, preferably is 55-75 kHz, more preferably is 60 kHz. The number of the first transducer 11 is one, and the number of the second transducers 12 is 6. The second transducers 12 are in a circle, and the first transducer 11 is in the center of the circle.

### Forth Embodiment

In this embodiment (FIG. 23), the ultrasound fat reduction equipment for cavitating adipose tissue and for treating adipose in a region of interest comprises a movable transducer, two rods, some sensors and a first handpiece 3. The movable transducer is configured to slide along the rods by electronical control to change the distance between the movable transducer and the region of interest.

The sensors are wired pressure sensors 4 (they can be wired temperature sensors, or can be wired pressure sensors and wired temperature sensors, in some embodiments). The detection interface of the wired pressure sensors 4 are flush with the outer surface of the treatment head 31 of the first handpiece. The wire of the wired pressure sensors 4 gets through the tail 32 of the first handpiece to connect to the control and data processing module of the ultrasound fat reduction equipment.

### Fifth Embodiment

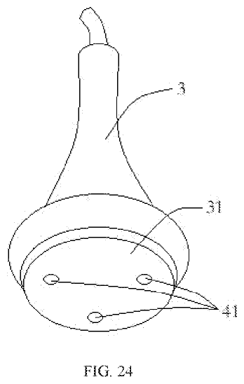

In this embodiment (FIG. 24), the ultrasound fat reduction equipment for cavitating adipose tissue and for treating adipose in a region of interest comprises a movable transducer, two rods, some sensors and a first handpiece 3. The movable transducer is configured to slide along the rods by electronical control to change the distance between the movable transducer and the region of interest.

The sensors are wireless pressure sensors 41 (they can be wired temperature sensors or can be wired pressure sensors and wired temperature sensors, in some embodiments). The wireless pressure sensors 41 are membrane structures that are applied to the outer surface of the treatment head 31 of the first handpiece. The wireless pressure sensors 41 use blue tooth to exchange data with the control and data processing module of the ultrasound fat reduction equipment.

### Sixth Embodiment

In this embodiment, the sensors are wireless pressure sensors (they can be wired temperature sensors, or can be wired pressure sensors and wired temperature sensors, in some embodiments). The wireless pressure sensors are membrane structures that are applied to the outer surface of the treatment head of the first handpiece. The wireless pressure sensors use blue tooth to exchange data with the control and data processing module of the ultrasound fat reduction equipment. The rest is the same as in the first embodiment.

### Seventh Embodiment

In this embodiment, the method of using the ultrasound fat reduction equipment disclosure herein comprises using coupling medium to fill the gap between a treatment head of the first handpiece and the region of interest.

The coupling medium comprises composition A. The composition A includes water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol and methyl hydroxybenzoate. The composition A further includes xanthan gum, dipotassium glycyrrhizinate, essence, ethylhexylglycerol, EDTA disodium and glucose as trace components.

### Eighth Embodiment

In this embodiment, the coupling medium further comprises composition B. The composition B includes water, propylene glycol, glycerin, carbomer, triethanolamine, p-hydroxyacetophenone, allantoin, dipotassium glycyrrhizinate, ribonucleic acid and 1,2-pentanediol. The composition B further includes hydroxyethylcellulose, octyl hydroxamic acid, sodium hyaluronate, punica granatum peel extract, nonylphenol polyether-15, butanediol, essence, nonylphenol polyether-10, camellia sinensis extract, glycerol octanoate, ellagic acid, galla thois extract, 1,2-hexanediol, glutathione, polysorbate-60, disodium hydrogen phosphate, acetylhexapeptide-8 and sodium dihydrogen phosphate as trace components. The rest is the same as in seventh embodiment.

### Ninth Embodiment

In this embodiment, the coupling medium further comprises composition C. The composition C includes mineral oil, vitis vinifera seed oil, helianthus annus seed oil, citrus aurantium dulcis peel oil, tocopherol acetate and hibiscus abelmoschus seed extract. The rest is the same as in seventh embodiment.

While only certain features and embodiments of the invention have been illustrated and described herein, many modifications and changes will occur to those skilled in the art. It is, therefore, to be understood that the appended claims are intended to cover all such modifications and changes as fall within the scope of the invention.

1. An ultrasound fat reduction equipment for cavitating adipose tissue and for treating adipose in a region of interest, comprising:
an input module for inputting data; a storage module for storing data from the input module;
a control and data processing module for controlling the generation of ultrasound which comprising turning on and turning off ultrasound, adjusting energy levels, and setting treatment durations, and processing data from the input module and the storage module; and
a machine learning module for using the data from the storage module to learn to generate a recommended best treatment procedure for a certain input data panel, and transferring to the control and data processing module to output the recommended best treatment procedure; the input module, the storage module, and the machine teaming module all communicate with the control and data processing module

2. The ultrasound fat reduction equipment of claim 1, further comprising one or more sensors, which is/are on the outer surface of the top of the ultrasound fat reduction equipment's handpiece; the sensor(s) is/are a pressure sensor, and/or a temperature sensor; the data using for the machine learning module includes individual characteristics data, treatment data, sensor data and the relationship of these 3 kinds of data.

3. The ultrasound fat reduction equipment of claim 2, wherein the individual characteristics data includes at least 1, 2, 3, 4, 5, 6, 7, 8, 9, 10, or all 11 of age, gender, weight, BMI, waist measurement, fat content in %, fat layer thickness, density of the fat in the treatment area, degree of skin tightness or looseness, and ultrasound images; the treatment data includes at least 1, 2, 3, or all 4 of energy levels, treatment durations, ultrasound frequencies and treatment outcome data; the sensor data includes data from the pressure sensor, and/or data from the temperature sensor.

4. The ultrasound fat reduction equipment of claim 1, wherein the machine learning module is configured to generate a recommended best treatment procedure through an Al model of optimized ultrasound cavitation treatment parameters.

5. The ultrasound fat reduction equipment of claim 4, wherein the modelling method of the Al model of optimized ultrasound cavitation treatment parameters comprises: initial Al model construction: selecting suitable Al algorithms, such as machine learning algorithms or deep learning neural networks, and train the model using the training set, and depending on the dataset and objectives, supervised learning, unsupervised learning, deep learning, reinforcement learning, or causal inference approaches being employed to construct an initial Al model.

6. The ultrasound fat reduction equipment of claim 5, wherein causal inference involves propensity score matching, and/or instrumental variables, and/or causal graphical models.

7. The ultrasound fat reduction equipment of claim 5, wherein the modelling method of the Al model of optimized ultrasound cavitation treatment parameters further comprises: parameter optimization: using the initial Al model and training dataset for parameter optimization to find the optimal ultrasound cavitation treatment parameters, which can be achieved through optimization algorithms such as gradient descent, genetic algorithms, or Bayesian optimization.

8. The ultrasound fat reduction equipment of claim 1, further comprising a movable transducer configured to generate ultrasound-waves to cavitate fat cells and a first handpiece, and the movable transducer can move vertically inside the first handpiece, in order to change the distance between the movable transducer and the region of interest.

9. The ultrasound fat reduction equipment of claim 8, further comprising one or more rods inside the first handpiece, wherein the movable transducer is configured to slide along these rod(s) through either a mechanical, which is manipulated on the surface of the first handpiece on the surface of the first handpiece, or through electronic control to change the distance between the movable transducer and the region of interest.

10. The ultrasound fat reduction equipment of claim 8, wherein the movable transducer comprises: a first transducer configured to generate ultrasound waves with a lower frequency, in the range of 25-50 kHz, preferably is 25-45 kHz, 30-45 kHz, or 30-40 kHz, more preferably is 35 kHz or 40 kHz; and/or a second transducer configured to generate ultrasound waves with a higher frequency, in the range of 50-90 kHz, preferably is 55-90 kHz, 55-85 kHz, 55-75 kHz, or 55-70 kHz, more preferably is 60 kHz.

11. The ultrasound fat reduction equipment of claim 8, further comprising one or more sensors situated on the outer surface of the top of the first handpiece; the sensor(s) is/are selected from a pressure sensor, a temperature sensor.

12. The ultrasound fat reduction equipment of claim 8, wherein the skin contacting end of the first handpiece's surface is textured with certain patterns, which are selected from closed design, open design and mixed design with both closed design and open design.

13. The ultrasound fat reduction equipment of claim 12, wherein the surface of the skin contacting end is raised, preferably the center of the surface is raised, more preferably the center of the surface is gradually raised.

14. The ultrasound fat reduction equipment of claim 12, wherein the surface of the skin contacting end is concave, preferably with a concavity in the center of the surface, more preferably with a gradual concavity in the center of the surface.

15. The ultrasound fat reduction equipment of claim 8, further comprising a second handpiece, which can produce ultrasound waves using for ultrasound imaging.

16. A method of using the ultrasound fat reduction equipment of claim 1 comprising: inputting data which includes individual characteristics data; comparing the individual characteristics data with the data from a databank to generate a recommended best treatment procedure; wherein the databank is a part of the machine learning module and includes training set data and validation set data.

17. The method of claim 16, further comprising: using coupling medium to fill the gap between a treatment head of the first handpiece and the region of interest; wherein said coupling medium is selected from composition A, and/or composition B, and/or composition C, and/or composition D; the composition A includes at least 1, 2, 3, 4, 5, 6, 7, 8, 9, 10, 11, 12, 13, 14 or all 15 of water, mineral oil, cetearyl alcohol, PEG-8, glycerol stearate, glycerin, PEG-100 stearate, cyclopentamethylene siloxane, cyclohexane siloxane, carbomer. triethanolamine, allantoin, cetearyl glucoside, phenoxyethanol, and methyl hydroxy benzoate; the composition B at least 1, 2, 3, 4, 5, 6, 7, 8, 9, or all 10 of includes water, propylene glycol, glycerin, carbomer, triethanolamine, p-hydroxyacetophenone, allantoin, dipotassium glycyrrhizinate, ribonucleic acid, and 1,2-pentanediol; the composition C includes at least 1, 2, 3, 4, 5, or all 6 of mineral oil, vitis vimfera seed oil, helianthus annus seed oil, citrus aurantium dulcis peel oil, tocopherol acetate, and hibiscus abelmoschus seed extract; the composition D includes mRNA, and/or microRNA, and/or antisense RNA for collagen production.

18. The method of claim 16, wherein the composition A further includes at least 1, 2, 3, 4, 5, or all 6 of xanthan gum, dipotassium glycyrrhizinate, essence, ethylhexylglycerol, EDTA disodium, and glucose.

19. A computer-readable medium, characterized in that it comprises one or more processors for causing the processor to perform operations of the method of claim 16.

20. An electronic device, characterized in that it comprises: a processor, and a memory, the processor being connected to the memory; the memory for storing a computer program of the processor, wherein the processor is configured to implement the method described in claim 16 by executing the computer program.
